(19) United States
(12) Patent Application Publication
(10) Pub. No.: US 2014/0295483 A1
(43) Pub. Date: Oct. 2, 2014

(54) APPARATUS AND METHOD FOR IN-VITRO DRUG SCREENING FOR CANCER METASTASIS

(71) Applicant: Board of Regents, The University of Texas System, Austin, TX (US)

(72) Inventors: Aaron B. Baker, Austin, TX (US); Christopher Spruell, Pflugerville, TX (US)

(73) Assignee: Board of Regents, The University of Texas System, Austin, TX (US)

(21) Appl. No.: 14/223,481

(22) Filed: Mar. 24, 2014

Related U.S. Application Data
(60) Provisional application No. 61/807,180, filed on Apr. 1, 2013.

(51) Int. Cl. G01N 33/50

(52) U.S. Cl. CPC G01N 33/5011
USPC 435/32; 435/288.4

(57) ABSTRACT

The present invention includes an apparatus and methods for detecting cancer cell metastasis, the apparatus comprising: a multi-well plate for growing cells in tissue culture; one or more shafts, cones, or fins that fit within one or more of the wells of the multi-well plate and that can be positioned in close proximity to the bottom of the one or more wells; a rotational motor connected to drive the one or more shafts, cones, or fins to rotate, such that rotation of the shafts, cones, or fins creates fluid flow within the one or more wells; and a detector capable of measuring cells within the wells.

### CROSS-REFERENCE TO RELATED APPLICATIONS

This application claims priority to U.S. Provisional Patent Application Ser. No. 61/807,180, filed Apr. 1, 2013.

### TECHNICAL FIELD OF THE INVENTION

The present invention relates in general to the field of detecting cellular metastasis, and more particularly, to an apparatus and method for measuring cell adhesion to screen for cancer metastasis.

### STATEMENT OF FEDERALLY FUNDED RESEARCH

None.

### INCORPORATION-BY-REFERENCE OF MATERIALS FILED ON COMPACT DISC

None.

### BACKGROUND OF THE INVENTION

Without limiting the scope of the invention, its background is described in connection with cancer metastasis.

Advances in the successful treatment of cancer by existing approaches such as surgery and chemotherapy remain limited by the presence of metastatic disease. A recent estimate suggests that over 90% of cancer deaths are attributable to the presence of metastases distant from the primary tumor. In order to metastasize, tumor cells must undergo a series of steps. They must invade the lymphatic or blood vessels (intravasation), survive while traveling through these vessels, and finally migrate out into a new location in the body (extravasation). While these processes are critical determinants of the aggressiveness of a cancer and patient survival, the majority of studies and drug screening assays performed examine, instead, tumorigenesis or tumor growth. Thus, a fundamental limitation in the development of urgently-needed drugs for the treatment of metastatic cancers is a lack of high-throughput assays for compounds targeting the fundamental steps of cancer cell metastasis. The challenge in this area is that in vitro tests for cancer metastasis lie outside the traditional paradigms for high-throughput assays and, by their nature, must contain cancer cells in suspension moving under physiologic flow over a monolayer of vascular or lymphatic cells.

### SUMMARY OF THE INVENTION

In one embodiment, the present invention includes an apparatus comprising: a multi-well plate for growing cells in tissue culture; one or more shafts, cones, or fins that fit within one or more of the wells of the multi-well plate and that can be positioned in close proximity to the bottom of the one or more wells; a rotational motor connected to drive the one or more shafts, cones, or fins to rotate, such that rotation of the shafts, cones, or fins creates fluid flow within the one or more wells; and a detector capable of measuring cells within the wells. In one aspect, the one or more tips of the one or more shafts are shaped to provide fluid flow on or about cells that are grown in the wells of the multi-well plate. In another aspect, the detector is a laser speckle imager capable of quantifying the velocity of fluid at or about the bottom of the one or more well. In another aspect, the apparatus further comprises a computer connected to at least one of the rotational motor, the detector, or both, and that includes one or more code segments that: calculate the fluid flow within each of the one or more wells; that measure cell death on exposure to fluid flow; or that measure cell adhesion. In another aspect, the detector is capable of counting the number of cells within each well during at least one of before, during, or after rotation of the shafts to cause fluid flow in the wells. In another aspect, the cells are grown in the wells, and the cells are human cancer cells. In another aspect, the one or more shafts are capable of fluid flow within the well of between 0.1 dyn/cm² to 20 dyn/cm², 0.5 dyn/cm² to 15 dyn/cm², 1.0 dyn/cm² to 12 dyn/cm², 0.2 dyn/cm² to 12 dyn/cm², and 5.0 dyn/cm² to 12 dyn/cm². In another aspect, the flow is an oscillatory flow. In another aspect, the apparatus further comprises a computer connected to the detector, wherein the computer calculates at least one of cell adhesion, cell death, cell size, cell morphology, or cell movement, and wherein changes in cell adhesion, cell death, cell size, cell morphology, or cell movement are indicative of cellular metastasis. In another aspect, the device is sized to be inserted into a well of a multi-well plate selected from the group consisting of 2, 4, 6, 8, 10, 12, 24, 48, 96, 394, and 1536 well plates. In another aspect, the shafts are selected from at least one of biocompatible material, sterile, smooth, rough, trapezoidal, conical, flat, cut, or polygonal. In another aspect, the shafts are at least one of steel, steel alloy, stainless steel, titanium, plastic, polymer, glass, quartz, or wood. In another aspect, the detector is at least one of plate reader, a cell sorter, a cell counter, a microscope, or a camera. In another aspect, the multi-well plate comprises a polyacrylate, a polymethylacrylate, a polycarbonate, a polysulphone, a polyhydroxy acid, a polyanhydride, a polyorthoester, a polypropylene, a polyphosphazene, a polyphosphate, a polyester, a nylon or a mixture thereof. In another aspect, the wells are defined further as comprising a first chamber and a second chamber, wherein the chambers are separated by a membrane sized to allow cellular transit between the first and second chambers if cells are metastatic.

Another embodiment of the present invention includes a method of detecting cellular metastasis comprising: providing a multi-well plate comprising a bottom surface and one or more rotatable shafts, cones, or fins positioned within one or more of the wells in close proximity to the bottom of the one or more wells, wherein the shafts, cones, or fins are rotated by a rotational motor connected to drive the one or more shafts, cones, or fins to rotate, such that rotation of the shafts creates fluid flow within the one or more wells; growing one or more cells in the wells under different physiological fluid flow conditions; and detecting the motility of the one or more cells within the wells, wherein changes in cellular motility are indicative of metastasis. In another aspect, the one or more tips of the one or more shafts are shaped to provide fluid flow on or about cells that are grown in the wells of the multi-well plate. In another aspect, the detector is a colorimetric, fluorimetric, optical density, light scattering, laser, laser speckle imager capable of quantifying the velocity of fluid at or about the bottom of the one or more well. In another aspect, the method further comprises a computer connected to at least one of the rotational motor, the detector, or both, and that includes one or more code segments that: calculate the fluid flow within each of the one or more wells; that measure cell death on exposure to fluid flow; or that measure cell adhesion. In another aspect, the detector is capable of counting the number of cells within each well during at least one of before, during, or after rotation of the shafts to cause fluid flow in the wells. In another aspect, the cells are grown in the wells, and the cells are human cancer cells. In another aspect, the fluid flow is between 0.1 dyn/cm² to 20 dyn/cm², 0.5 dyn/cm² to 15 dyn/cm², 1.0 dyn/cm² to 12 dyn/cm², 0.2 dyn/cm² to 12 dyn/cm², and 5.0 dyn/cm² to 12 dyn/cm². In another aspect, the flow is an oscillatory flow. In another aspect, the method further comprises a computer connected to the detector, wherein the computer calculates at least one of cell adhesion, cell death, cell size, cell morphology, or cell movement, and wherein changes in cell adhesion, cell death, cell size, cell morphology, or cell movement are indicative of cellular metastasis. In another aspect, the device is sized to be inserted into a well of a multi-well plate selected from the group consisting of 2, 4, 6, 8, 10, 12, 24, 48, 96, 394, and 1536 well plates. In another aspect, the shafts are selected from at least one of biocompatible material, sterile, smooth, rough, trapezoidal, conical, flat, cut, or polygonal. In another aspect, the shafts are at least one of steel, steel alloy, stainless steel, titanium, plastic, polymer, glass, quartz, or wood. In another aspect, the detector is at least one of plate reader, a cell sorter, a cell counter, a microscope, or a camera. In another aspect, the multi-well plate comprises a polyacrylate, a polymethylacrylate, a polycarbonate, a polysulphone, a polyhydroxy acid, a polyanhydride, a polyorthoester, a polypropylene, a polyphosphazene, a polyphosphate, a polyester, a nylon or a mixture thereof. In another aspect, the wells are defined further as comprising a first chamber and a second chamber, wherein the chambers are separated by a membrane sized to allow cellular transit between the first and second chambers if cells are metastatic.

Another embodiment of the present invention includes a kit for conducting in-vitro assays for determining cellular adhesion comprising: one or more multi-well plates for growing cells in tissue culture; one or more shafts, cones, or fins that fit within one or more of the wells of the multi-well plate and that can be positioned in close proximity to the bottom of the one or more wells connected to a rotational motor connected to drive the one or more shafts, cones, or fins to rotate, such that rotation of the shafts, cones, or fins creates fluid flow within the one or more wells; and optionally a detector capable of measuring cells within the wells.

Another embodiment of the present invention includes a method of detecting cellular metastasis comprising: providing a multi-well plate comprising a bottom surface and one or more rotatable shafts, cones, or fins positioned within one or more of the wells in close proximity to the bottom of the one or more wells, wherein the shafts, cones, or fins are rotated by a rotational motor connected to drive the one or more shafts, cones, or fins to rotate, such that rotation of the shafts creates fluid flow within the one or more wells; obtaining a patient sample comprising cells suspected of having metastatic cancer; growing the cells in the wells under different physiological fluid flow conditions; and detecting the motility of the one or more cells within the wells, wherein changes in cellular motility are indicative of metastasis.

Another embodiment of the present invention includes a method for evaluating a candidate drug believed to be useful in treating a metastatic cancer in vitro, the method comprising: contacting a candidate drug in vitro to a first subset of metastatic cancer cells and a placebo to a second subset of metastatic cancer cells; providing a multi-well plate comprising a bottom surface and one or more rotatable shafts, cones, or fins positioned within one or more of the wells in close proximity to the bottom of the one or more wells, wherein the shafts, cones, or fins are rotated by a rotational motor connected to drive the one or more shafts, cones, or fins to rotate, such that rotation of the shafts creates fluid flow within the one or more wells; growing the first and the second subset of cells in the wells under different physiological fluid flow conditions; and detecting the motility of the first and the second subset of cells within their respective wells; repeating the step of detecting the motility of the cells from the first and the second subset of cells; and determining if the candidate drug reduces cancer metastasis from the first subset of cells that is statistically significant as compared to any reduction occurring in the second subset of cells, wherein a statistically significant reduction indicates that the candidate drug is useful in treating the metastatic cancer cells.

Another embodiment of the present invention includes a method of performing a clinical trial to evaluate a candidate drug believed to be useful in treating a metastatic cancer, the method comprising: administering a candidate drug to a first subset of the patients, and a placebo to a second subset of the patients; providing a multi-well plate comprising a bottom surface and one or more rotatable shafts, cones, or fins positioned within one or more of the wells in close proximity to the bottom of the one or more wells, wherein the shafts, cones, or fins are rotated by a rotational motor connected to drive the one or more shafts, cones, or fins to rotate, such that rotation of the shafts creates fluid flow within the one or more wells; growing one or more cells from the first and subset of the patients in the wells under different physiological fluid flow conditions; and detecting the motility of the one or more cells within the wells from the first and subset of the patients, repeating step of administration of the candidate drug or the placebo; and repeating the step of detecting the motility of the cells from the first and subset of the patients; and determining if the candidate drug reduces cancer metastasis from the first set of patients that is statistically significant as compared to any reduction occurring in the second subset of patients, wherein a statistically significant reduction indicates that the candidate drug is useful in treating the cancer.

### BRIEF DESCRIPTION OF THE DRAWINGS

For a more complete understanding of the features and advantages of the present invention, reference is now made to the detailed description of the invention along with the accompanying figures and in which:

FIG. 1 shows that a rotational motor drives the shafts to rotate and the close proximity of the cone to the cell culture surface creates flow within the well plate.

FIG. 2 shows the apparatus of the present invention.

FIG. 3 is an image that shows the shafts interacting with the culture plate and used laser speckle imaging to quantify the velocity at the cell culture surface.

FIG. 4 shows a computational model of the flow within the system that allows us to predict and control the flow profiles applied to circulating cells in the system.

FIG. 5 shows the results from biocompatibility studies measured cell death on exposure to flow also confirmed very low cell death during application of flow.

FIG. 6 shows the adherence of the leukemia cells using the system of the present invention.

FIG. 7 shows that certain cells were highly affected by the presence of physiological flow but slightly affected by the inflammation of endothelial cells.

FIG. 8 shows an adaptation of the present invention for use with cancer cell invasion across a membrane.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

The present invention allows the application of a user-defined shear stress to cells in a wide variety of multi-well plate formats, e.g., 2, 4, 6, 8, 10, 12, 24, 48, 96, 394, or 1536-well culture plate. In the 96-well plate configuration, the device includes up to 96 rotating rods with, e.g., coned tips, interconnected at one end via a gearbox. The gearbox is connected to a servomotor via, e.g., an Oldham coupling driveshaft. When submerged in a fluid such as cell culture media, each individual cone tipped rod rotates to produce a shear stress proportional to the angular velocity. The user can program the motor to preform a wide array of movements by using a GUI on a laptop computer connected to the motor and controller via USB. A micrometer resolution lift stage below the rotating cone tipped rods provides a platform on which to secure a standard 96-well culture plate. The plate is then raised (or the shafts are lowered) such that each rod enters each individual well. The mechanical apparatus is then placed inside an incubator and the desired experiment is run.

Unlike existing systems, the apparatus of the present invention provides an additional layer of control into existing cell culture systems. Specifically, it allows the control of shear stress on a number of important physiological functions in a high throughput manner that is amenable to integration with robotic cell culture and drug screening technologies.

Another advantage of this system is its high-throughput nature. Previous approaches utilize a single cone tip allowing the experimenter to conduct only a single experiment. This apparatus of the present invention allows parallel studies in a consistent manner in each well, thus, allowing the researcher to preform multiple studies, study conditions, cells, titrations, etc., in parallel. In addition, the apparatus of the present invention contains a height adjustable stage capable of micrometer accuracy. This stage allows the distance between the cells and the rotating cone to be precisely controlled. Computer models indicate that adjusting this distance can create defined shear stress gradients in the bottom of each well. Current research indicated that these gradients play an important role in many physiological and pathophysiological functions.

Current cell culture techniques are largely static and do not allow for the consideration of important physiological functions such as fluid flow. A fluid moving across a stationary boundary creates a shear stress. In living organisms, moving fluids such as blood create shear stresses that regulate important biological functions. Modern cell culture techniques strive to mimic physiological conditions as closely as possible however they fail to provide a way to apply a fluid shear stress to cells in the culture environment. In order to study these important phenomena, scientists must often resort to expensive and unpredictable animal models. The present inventors have developed a system that allows scientists to integrate an additional layer of control into their existing cell culture setups by allowing for the precise and defined application of a fluid shear stress. In such a setup, the scientist may simply insert a cell culture dish into the apparatus and implement a program to expose the cells to his defined conditions. Studies on shear stress are imperative in the flowing active areas of research.

### Cardiovascular Research Drug Discovery

Cardiovascular disease remains the number one killer of Americans. Studies indicate that fluid shear stresses are involved in a number of important cardiovascular functions including: coagulation/thrombosis, platelet aggregation and formation of atherosclerotic plaque. This system would be useful as a high throughput assay for studying the effects of shear stress on cardiovascular/vascular cell types and for studying or diagnosing diseases or syndromes involving blood coagulation of thrombosis. In addition, the rupture of these plaques has been shown to be dependent of shear stress and often results in cardiac arrest.

### Cancer Research/Drug Discovery

Malignant neoplasms are the second most common killer of Americans. Research indicates that the metastasis of cancers such as colon and breast are shear stress sensitive. Cancer cells use the vascular and lymphatic circulatory systems to spread throughout the body. In these systems they are exposed to shear stress/fluid flow. In addition, circulating tumor cells (CTCs) are cancer cells shed by tumors in to the circulatory system and are potential markers for diagnosing and guiding the treatment of cancer. The apparatus of the present invention may be used to study the effects of cancer metastasis under a controllable shear stress to aid in the development of drugs or create a novel diagnostic tool.

### Immune System Function

The apparatus of the present invention is capable of allowing researchers to make discoveries about the human immune system. By using this apparatus with the addition of floating leucocytes in the cell culture media, researchers may investigate the effects of immune cell adhesion under a variety of conditions or chemical/drug treatments. In addition, the system would allow the high throughput study of adhesion and invasion of pathogens such as viruses, bacteria or parasites during their interaction adherent cells (e.g., intestinal/lung epithelial cells or other cell type exposed to pathogens).

These active areas of industrial and academic research would benefit from this device as it would allow researchers to conduct a large number of studies in timely/efficient manner and interfacing with many standard well-plate assays or robotic pipetting/tissue culture systems as well as automated high content screening systems that have integrated cell counting and other advanced functions.

Additionally, this system is capable of generating spatial gradients in shear stress, whereas current technologies cannot. Finally, this system is capable of incorporating cell in suspension in the cell culture media surrounding the rod. This setup can be used to study the interactions of suspended cancer cells or immune cells with an adherent cell type such as endothelial cells and lymph endothelial cells as a means to screen for drugs to stop cancer metastasis or inflammation.

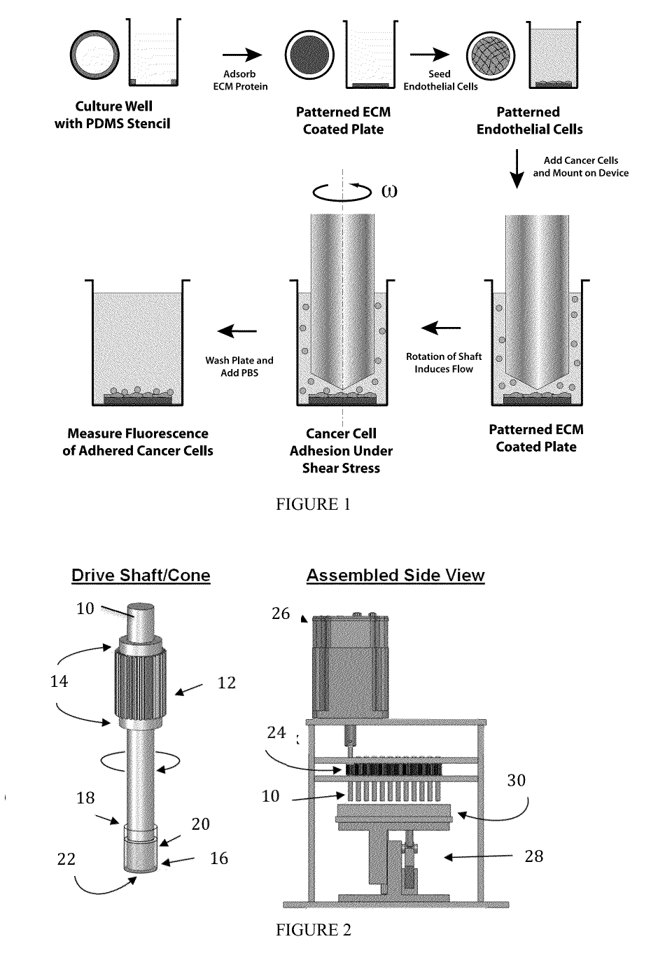

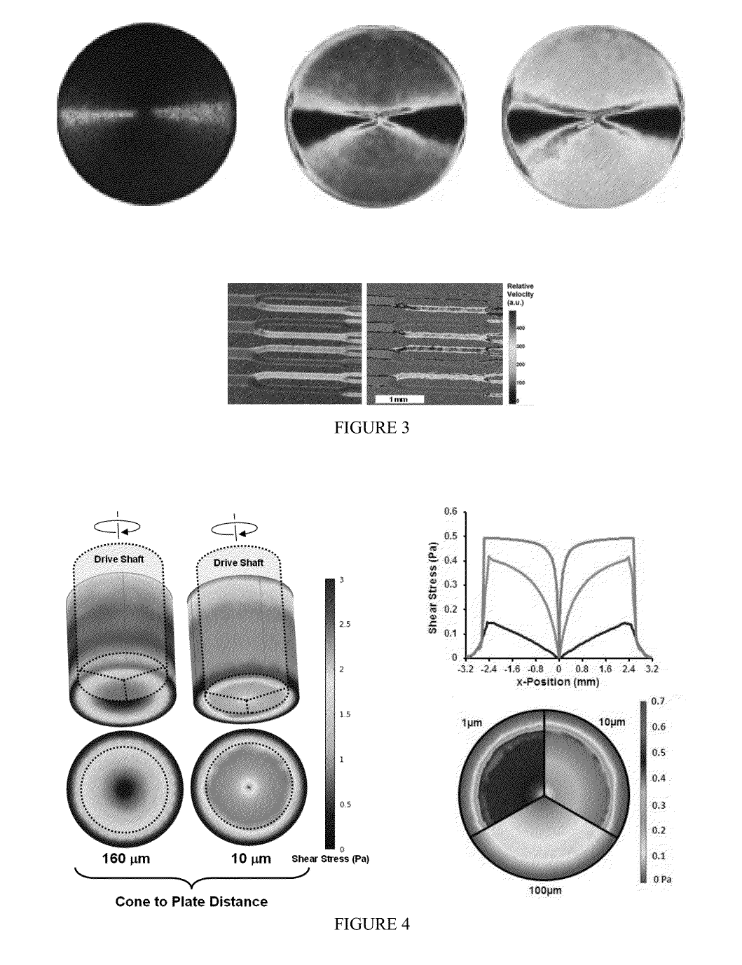

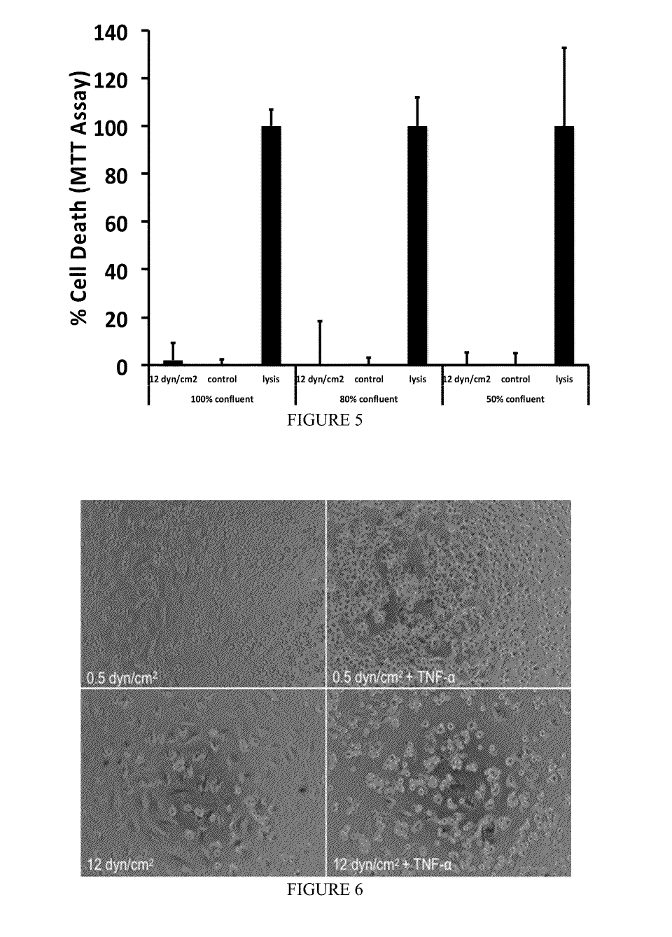

The present inventors have designed, built and validated a high-throughput multi-well assay that allows the study of cancer metastasis under different physiological flow conditions. This device applies flow to cells in culture through the rotation of 96 cone-tipped shafts linked through interfacing gears. A rotational motor drives the shafts to rotate and the close proximity of the cone to the cell culture surface creates flow within the well plate (FIG. 1). The completed device is shown in FIG. 2. The left side of FIG. 2 shows a drive shaft/cone 10 is shown that includes a gear 12 with bearings 14 that can be rotated in either direction or in an oscillatory or other manner, that is shown having a tip 16 (e.g., a shaft, cone, fin or paddle) portion in a cell culture well 18 having a media 20 and cells 22. The right image in FIG. 2 shows multiple drive shaft/cone 10 attached to a gear box 24, which is connected to a motor 26. A lift table 28 (e.g., a micrometer lifting table) lowers and raised a plate 30 (shown here as a 96-well plate), with shafts in at least one row, one column, a portion or all of wells of the plate. The apparatus of the present invention was used to image the shafts (shows as cones) interacting with the culture plate and used laser speckle imaging to quantify the velocity at the cell culture surface (FIG. 3). In addition, a computational model was created of the flow within the system (FIG. 4) that allows the inventors to predict and control the flow profiles applied to circulating cells in the system. Biocompatibility studies measured cell death on exposure to flow was also confirmed very low cell death during application of flow (FIG. 5).

Next, several assays were performed to demonstrate the system's ability to measure cancer cell adhesion. Cultured cell isolated from a human patient with monocytic leukemia (THP-1 cells) were used. THP-1 cells are leukemia cell derived from the white blood cells of the body. Even though these are cancer cells, they also retain some of the properties of white blood cells including responding to inflammatory cytokines such as TNF-α. The inventors stimulated endothelial cells grown in a 96-well plate with 10 ng/ml TNF-α for 1 hour. They then circulated the fluorescently labeled THP-1 cells over endothelial cells using the system of the present invention with shear stress of 0.5 dynes/cm² for 10 minutes. The wells were then wash with buffer and the plates assayed for fluorescent cell attachment using a fluorescence plate reader or epifluorescent microscope (FIG. 6).

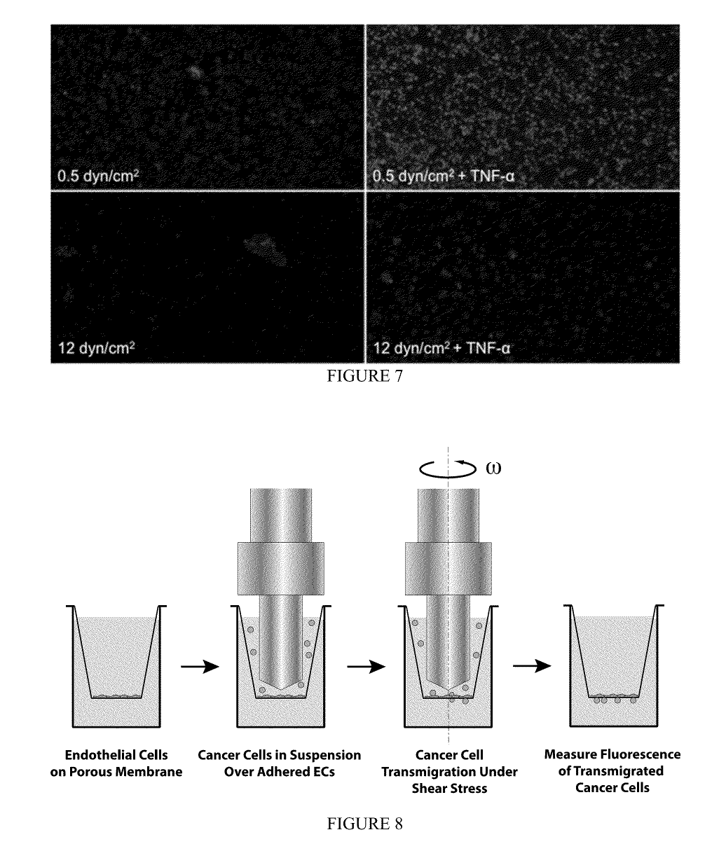

In a separate study, colon cancer cells derived from patients with cancer of varying aggressiveness were used. Four different types of colon cancer cells were placed in contact with endothelial cells under different flow conditions: static (0 dyn/cm²), low (0.5 dyn/cm²), and normal (12 dyn/cm²) flow. Endothelial cells grown in a 96-well plate were treated with TNF-α to induce inflammation. The effect of flow and inflammation on adhesion of cancer cells was cell dependent; however, all cells were affected by the presence of flow or inflammation. For example, adhesion of RKO1 cells was highly affected by the presence of inflammation and flow. On the other side, DLD-1 cells were highly affected by the presence of physiological flow but slightly affected by the inflammation of endothelial cells (FIG. 7). This study shows for the first time the effects of inflammation on the adherence of circulating cancer cells. In addition, they provide validation that the system can distinguish between aggressive and nonaggressive cancers. Another key aspect of the metastatic process is the invasion of cancer cells into the tissue after adhesion to the vascular or lymphatic endothelium (a process known as extravasation). By replacing the standard 96-well plate with a 96-well plate with a porous cell culture surface the system can be adapted to be able to measure both the adhesion and migration through a confluent endothelial/lymphendothelial layer. FIG. 8 shows an adaptation of the present invention for use with cancer cell invasion across a membrane.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context. In certain embodiments, the present invention may also include methods and compositions in which the transition phrase “consisting essentially of” or “consisting of” may also be used.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

What is claimed is:

1. An apparatus comprising:
a multi-well plate for growing cells in tissue culture;
one or more shafts, cones, or fins that fit within one or more of the wells of the multi-well plate and that can be positioned in close proximity to the bottom of the one or more wells;
a rotational motor connected to drive the one or more shafts, cones, or fins to rotate, such that rotation of the shafts, cones, or fins creates fluid flow within the one or more wells; and
a detector capable of measuring cells within the wells.

2. The apparatus of claim 1, wherein one or more tips of the one or more shafts are shaped to provide fluid flow on or about cells that are grown in the wells of the multi-well plate.

3. The apparatus of claim 1, wherein the detector is a laser speckle imager capable of quantifying the velocity of fluid at or about the bottom of the one or more well.

4. The apparatus of claim 1, further comprising a computer connected to at least one of the rotational motor, the detector, or both, and that includes one or more code segments that: calculate the fluid flow within each of the one or more wells; that measure cell death on exposure to fluid flow; or that measure cell adhesion.

5. The apparatus of claim 1, wherein the detector is capable of counting the number of cells within each well during at least one of before, during, or after rotation of the shafts to cause fluid flow in the wells.

6. The apparatus of claim 1, wherein cells are grown in the wells, and the cells are human cancer cells.

7. The apparatus of claim 1, wherein the one or more shafts are capable of fluid flow within the well of between 0.1 dyn/cm² to 20 dyn/cm², 0.5 dyn/cm² to 15 dyn/cm², 1.0 dyn/cm² to 12 dyn/cm², 0.2 dyn/cm² to 12 dyn/cm², and 5.0 dyn/cm² to 12 dyn/cm².

8. The apparatus of claim 1, wherein the flow is an oscillatory flow.

9. The apparatus of claim 1, further comprising a computer connected to the detector, wherein the computer calculates at least one of cell adhesion, cell death, cell size, cell morphology, or cell movement, and wherein changes in cell adhesion, cell death, cell size, cell morphology, or cell movement are indicative of cellular metastasis.

10. The apparatus of claim 1, wherein the device is sized to be inserted into a well of a multi-well plate selected from the group consisting of 2, 4, 6, 8, 10, 12, 24, 48, 96, 394, and 1536 well plates.

11. The apparatus of claim 1, wherein the shafts are selected from at least one of biocompatible material, sterile, smooth, rough, trapezoidal, conical, flat, cut, or polygonal.

12. The apparatus of claim 1, wherein the shafts are at least one of steel, steel alloy, stainless steel, titanium, plastic, polymer, glass, quartz, or wood.

13. The apparatus of claim 1, wherein the detector is at least one of plate reader, a cell sorter, a cell counter, a microscope, or a camera.

14. The apparatus of claim 1, wherein the multi-well plate comprises a polyacrylate, a polymethylacrylate, a polycarbonate, a polysulphone, a polyhydroxy acid, a polyanhydride, a polyorthoester, a polypropylene, a polyphosphazene, a polyphosphate, a polyester, a nylon or a mixture thereof.

15. The apparatus of claim 1, wherein the wells are defined further as comprising a first chamber and a second chamber, wherein the chambers are separated by a membrane sized to allow cellular transit between the first and second chambers if cells are metastatic.

16. A method of detecting cellular metastasis comprising:
providing a multi-well plate comprising a bottom surface and one or more rotatable shafts, cones, or fins positioned within one or more of the wells in close proximity to the bottom of the one or more wells, wherein the shafts, cones, or fins are rotated by a rotational motor connected to drive the one or more shafts, cones, or fins to rotate, such that rotation of the shafts, cones, or fins creates fluid flow within the one or more wells;
growing one or more cells in the wells under different physiological fluid flow conditions; and
detecting the motility of the one or more cells within the wells, wherein changes in cellular motility are indicative of metastasis.

17. The method of claim 16, wherein one or more tips of the one or more shafts are shaped to provide fluid flow on or about cells that are grown in the wells of the multi-well plate.

18. The method of claim 16, wherein the detector is a colorimetric, fluorimetric, optical density, light scattering, laser, laser speckle imager capable of quantifying the velocity of fluid at or about the bottom of the one or more well.

19. The method of claim 16, further comprising a computer connected to at least one of the rotational motor, the detector, or both, and that includes one or more code segments that: calculate the fluid flow within each of the one or more wells; that measure cell death on exposure to fluid flow; or that measure cell adhesion.

20. The method of claim 16, wherein the detector is capable of counting the number of cells within each well during at least one of before, during, or after rotation of the shafts to cause fluid flow in the wells.

21. The method of claim 16, wherein cells are grown in the wells, and the cells are human cancer cells.

22. The method of claim 16, wherein the fluid flow is between 0.1 dyn/cm² to 20 dyn/cm², 0.5 dyn/cm² to 15 dyn/cm², 1.0 dyn/cm² to 12 dyn/cm², 0.2 dyn/cm² to 12 dyn/cm², and 5.0 dyn/cm² to 12 dyn/cm².

23. The method of claim 16, wherein the flow is an oscillatory flow.

24. The method of claim 16, further comprising a computer connected to the detector, wherein the computer calculates at least one of cell adhesion, cell death, cell size, cell morphology, or cell movement, and wherein changes in cell adhesion, cell death, cell size, cell morphology, or cell movement are indicative of cellular metastasis.

25. The method of claim 16, wherein the device is sized to be inserted into a well of a multi-well plate selected from the group consisting of 2, 4, 6, 8, 10, 12, 24, 48, 96, 394, and 1536 well plates.

26. The method of claim 16, wherein the shafts are selected from at least one of biocompatible material, sterile, smooth, rough, trapezoidal, conical, flat, cut, or polygonal.

27. The method of claim 16, wherein the shafts are at least one of steel, steel alloy, stainless steel, titanium, plastic, polymer, glass, quartz, or wood.

28. The method of claim 16, wherein the detector is at least one of plate reader, a cell sorter, a cell counter, a microscope, or a camera.

29. The method of claim 16, wherein the multi-well plate comprises a polyacrylate, a polymethylacrylate, a polycarbonate, a polysulphone, a polyhydroxy acid, a polyanhydride, a polyorthoester, a polypropylene, a polyphosphazene, a polyphosphate, a polyester, a nylon or a mixture thereof.

30. The method of claim 16, wherein the wells are defined further as comprising a first chamber and a second chamber, wherein the chambers are separated by a membrane sized to allow cellular transit between the first and second chambers if cells are metastatic.

31. A kit for conducting assays for determining cellular adhesion comprising:
one or more multi-well plates for growing cells in tissue culture;
one or more shafts, cones, or fins that fit within one or more of the wells of the multi-well plate and that can be positioned in close proximity to the bottom of the one or more wells connected to a rotational motor connected to drive the one or more shafts, cones, or fins to rotate, such that rotation of the shafts, cones, or fins creates fluid flow within the one or more wells; and
optionally a detector capable of measuring cells within the wells.

32. A method of detecting cellular metastasis in vitro, comprising:
providing a multi-well plate comprising a bottom surface and one or more rotatable shafts, cones, or fins positioned within one or more of the wells in close proximity to the bottom of the one or more wells, wherein the shafts, cones, or fins are rotated by a rotational motor connected to drive the one or more shafts, cones, or fins to rotate, such that rotation of the shafts creates fluid flow within the one or more wells;
obtaining a patient sample comprising cells suspected of having metastatic cancer;
growing the cells in the wells under different physiological fluid flow conditions; and
detecting the motility of the one or more cells within the wells, wherein changes in cellular motility are indicative of metastasis.

33. A method for evaluating a candidate drug believed to be useful in treating a metastatic cancer in vitro, the method comprising:
contacting a candidate drug in vitro to a first subset of metastatic cancer cells and a placebo to a second subset of metastatic cancer cells;
providing a multi-well plate comprising a bottom surface and one or more rotatable shafts, cones, or fins positioned within one or more of the wells in close proximity to the bottom of the one or more wells, wherein the shafts, cones, or fins are rotated by a rotational motor connected to drive the one or more shafts, cones, or fins to rotate, such that rotation of the shafts creates fluid flow within the one or more wells;
growing the first and the second subset of cells in the wells under different physiological fluid flow conditions; and
detecting the motility of the first and the second subset of cells within their respective wells;
repeating the step of detecting the motility of the cells from the first and the second subset of cells; and
determining if the candidate drug reduces cancer metastasis from the first subset of cells that is statistically significant as compared to any reduction occurring in the second subset of cells, wherein a statistically significant reduction indicates that the candidate drug is useful in treating the metastatic cancer cells.

34. A method of performing a trial to evaluate a candidate drug believed to be useful in treating a metastatic cancer, the method comprising:
administering a candidate drug to a first subset of the patients, and a placebo to a second subset of the patients;
providing a multi-well plate comprising a bottom surface and one or more rotatable shafts, cones, or fins positioned within one or more of the wells in close proximity to the bottom of the one or more wells, wherein the shafts, cones, or fins are rotated by a rotational motor connected to drive the one or more shafts, cones, or fins to rotate, such that rotation of the shafts creates fluid flow within the one or more wells;
growing one or more cells from the first and subset of the patients in the wells under different physiological fluid flow conditions; and
detecting the motility of the one or more cells within the wells from the first and subset of the patients;
repeating the step of administration of the candidate drug or the placebo;
repeating the step of detecting the motility of the cells from the first and subset of the patients; and
determining if the candidate drug reduces cancer metastasis from the first set of patients that is statistically significant as compared to any reduction occurring in the second subset of patients, wherein a statistically significant reduction indicates that the candidate drug is useful in treating the cancer.
